SEQUENCE LISTING (4 sequences)

SEQ ID NO: 1 - DNA, 17 bp, Artificial Sequence
  Synthetic primer
ccaactgggacgacatg

SEQ ID NO: 2 - DNA, 17 bp, Artificial Sequence
  Synthetic primer
cctcgtagatgggcaca

SEQ ID NO: 3 - DNA, 20 bp, Artificial Sequence
  Synthetic primer
aaaggtttggtcctggcctt

SEQ ID NO: 4 - DNA, 20 bp, Artificial Sequence
  Synthetic primer
aaaggtttggtcctggcctt

(19) United States
(12) Patent Application Publication
(10) Pub. No.: US 2009/0054487 A1
(43) Pub. Date: Feb. 26, 2009

(54) Pharmaceutical composition for increasing the mitochondrial genesis

(76) Inventors: Attila Kolonics, Budapest (HU); Kalman Tory, Budapest (HU); Peter Literati Nagy, Budapest (HU); Zoltan Szilvassy, Debrecen-Jozsa (HU); Lajos Laszlo, Budapest (HU)

Correspondence Address: BIRCH STEWART KOLASCH & BIRCH, PO BOX 747, FALLS CHURCH, VA, 22040-0747, US

(21) Appl. No.: 11/629,279

(22) PCT Filed: Jun. 13, 2005
(86) PCT No.: PCT/IB2005/001639

§ 371 Date: Oct. 11, 2007

(30) Foreign Application Priority Data
Jun. 14, 2004 (HU) PO401177

(51) Int. Cl. A61K 31/4545; C07D 401/02

(52) U.S. Cl. 514/318; 546/193

(57) ABSTRACT

The invention refers to the use of O-(3-piperidino-2-hydroxypropyl)nicotinic amidoxime or a pharmaceutically suitable acid addition salt thereof for the preparation of a pharmaceutical composition increasing the mitochondrial genesis.

### FIELD OF THE INVENTION

The invention refers to a pharmaceutical composition for increasing the mitochondrial genesis. More specifically, the invention refers to the use of O-(3-piperidino-2-hydroxy-1-propyl)nicotinic amidoxime or a pharmaceutically suitable acid addition salt thereof for the preparation of a pharmaceutical composition that increases the mitochondrial genesis.

### BACKGROUND OF THE INVENTION

The mitochondrion is an essential organelle of the cell which occurs in varying number in the cytoplasm of every cell. That is the site of the cell's energy production. 98% of the oxygen used by the human organism is applied by the mitochondria for energy production. Oxidative phosphorylation taking place in the mitochondrion produces a considerable amount of ATP (adenosine triphosphate) that stores the energy needed by the cell. Thus, the number and state of mitochondrion is determinative from the point of view of life.

In function of physical requirement, the oxidative capacity of the striated muscle is able to change by an order of magnitude. The myofibrillar protein type of the muscle is changed and the mitochondrion content of the muscle is increased during accomodation to the load. In the regulation of mitochondrial function and formation, the transcription factor PGC-1α of the coactivator PPARγ (peroxisome proliferator-activated receptor γ) has key role. Mitochondrial biogenesis is also influenced by the calcium/calmoduline dependant kinase IV (CaMKIV), calcineurine, AMP-kinase [Zong H et al., Proc. Natl. Acad. Sci., 99, 15983 (2002)], MEF2 (myocyte enhancer factor 2), p38 MAPK as well as CREB, however, their effect is produced mainly through PGC-1α [Nisoli E. et al., Biochemical Pharmacology 67, 1 (2004)]. CAMKIV and calcineurin have an indirect influence on the activity of the promoter of PGC-1α, while p38 MAPK exerts its effect through the phosphorylation of PGC-1α and delaying the effect of the endogenic inhibiting domain [Fan M. et al., Genes & Development, 18, 278 (2004)]. According to recent observations, the nitric oxide produced by the endothelial nitric oxide synthase enzyme—through the increase of the activity of the guanilate cyclase enzyme and the cGMP level—plays a fundamental part in inducing the expression of PGC-1α and, thus, in the regulation of mitochondrial genesis [Nisoli, E., Science, 299, 896 (2003)].

Nitric oxide is a ubiquitous signal transducer molecule having very significant regulatory roles. Nitric oxide is produced from L-arginine by at least three different enzymes [neuronal nitric oxide synthase (nNOS), inducible nitric oxide synthase (iNOS) and endothelial nitric oxide synthase (eNOS)]. Neuronal type nitric oxide synthase is predominantly expressed in specific neurons of the brain, in non-adrenergic, non-cholinergic autonomic nerve cells, in muscles and in the macula densa region of the renal tubules, however, it is present at lower level in many other tissues as well. In the activation of nNOS enzyme, elevation of intracellular Ca⁺⁺ concentration and protein phosphorylation plays an immediate role. Furthermore, recent observations have revealed that the alteration of the expression level of the enzyme has a significant effect on the regulation of the activity thereof, too [Sasaki, M. et al., Proc. Natl. Acad. Sci. USA, 97, 8617 (2000)].

In addition to the energy production, the mitochondrion takes part also in the regulation of other physiological processes, for example, it is essential in the regulation and operation of the programmed cell death (apoptosis) [Martinou. J. C. és Green, D. R., Nat. Rev. Mol. Cell. Biol., 2, 63 (2001)].

Consequently, there are states and diseases when the number of mitochondria is insufficient relative to the requirement of the organism.

O-(3-piperidino-2-hydroxy-1-propyl)nicotinic amidoxime is known from U.S. Pat. No. 4,308,399. According to this document, the compound can be used for the treatment of diabetes angiopathy i.e. a vascular complication of diabetes.

It is know from U.S. Pat. No. 6,306,878 that hydroximic acid derivatives, especially the O-(3-piperidino-2-hydroxy-1-propyl)-nicotinic amidoxime, protect the mitochondrion from damages and can be employed for the treatment of diseases that develop through the damage of mitochondrion. The latter diseases include especially neurodegenerative ones such as Parkinson's disease and myopathies such as cardiomyopathy.

From WO 97/23198 it is known that O-(3-piperidino-2-hydroxy-1-propyl)nicotinic amidoxime protects the skin surface from the damaging effect of ultraviolet radiation, and the development of precancerous skin conditions can be inhibited by using the compound.

According to WO 98/58675 and WO 98/58676, O-(3-piperidino-2-hydroxypropyl)nicotinic amidoxime reduces the toxic side-effect of known antiviral and antitumor agents, respectively.

In accordance with WO 00/07580, O-(3-piperidino-2-hydroxypropyl)nicotinic amidoxime can be used for the treatment of autoimmune diseases such as type I diabetes mellitus (insulin-dependent diabetes mellitus, IDDM).

It is known from WO 03/007951 that O-(3-piperidino-2-hydroxypropyl)nicotinic amidoxime has an insulin sensitizing effect and enhances the effect of antidiabetic and anti-lipidemic agents on, among others, type II diabetes mellitus (non-insulin-dependent diabetes mellitus, NIDDM) and insulin resistance.

The aim of the invention is to produce a pharmaceutical composition promoting the mitochondrial genesis.

### SUMMARY OF THE INVENTION

It has been found that the above aim can be achieved by a pharmaceutical composition containing O-(3-piperidino-2-hydroxypropyl)nicotinic amidoxime or a pharmaceutically suitable acid addition salt thereof as the active agent. Surprisingly, in both in vitro cell culture and in vivo experiments it has been found that the latter active agent promotes the mitochondrial genesis.

### DESCRIPTION OF PREFERRED EMBODIMENTS

In accordance with our investigations, O-(3-piperidino-2-hydroxypropyl)nicotinic amidoxime or a pharmaceutically suitable acid addition salt thereof increases the expression and function of the nitric oxide synthase enzym both in vitro and in vivo. This effect can be the direct basis of the mitochondrial genesis inducing effect of the compound. According to the observation of Nisoli et al. cited above, through the signalization pathway NO-cGMP-PGC-1α, nitric oxide has a fundamental role in inducing the biogenesis of mitochondrion and in the regulation of the function of mitochondrion. Treatment with O-(3-piperidino-2-hydroxypropyl)nicotinic amidoxime or a pharmaceutically suitable acid addition salt thereof increases the nitric oxide concentration, thus, it can directly activate this regulation pathway through this mechanism. In addition to the number and function of mitochondria, the PGC-1α level regulates important metabolism pathways, too, thus, it has influence on the metabolism of the whole organism. Consequently, the O-(3-piperidino-2-hydroxypropyl)nicotinic amidoxime or a pharmaceutically suitable acid addition salt thereof may have an influence on the metabolism of the whole organism through the increase of PGC-1α level and mitochondrial number as well as the regulation of the key metabolic pathways.

Thus, the invention refers to the use of O-(3-piperidino-2-hydroxypropyl)nicotinic amidoxime or a pharmaceutically suitable acid addition salt thereof for the preparation of a pharmaceutical composition increasing the mitochondrial genesis.

O-(3-piperidino-2-hydroxy-1-propyl)nicotinic amidoxime can be prepared according to the process described in U.S. Pat. No. 6,306,878. A salt formed with an inorganic acid such as hydrochloric acid, sulfuric acid etc. or with an organic acid such as acetic acid, lactic acid, tartaric acid etc. can be used as a pharmaceutically suitable acid addition salt. Preferred acid addition salt of O-(3-piperidino-2-hydroxy-1-propyl)nicotinic amidoxime is the dihydrochloride thereof.

The effect of O-(3-piperidino-2-hydroxy-1-propyl)nicotinic amidoxime (the compound is also mentioned in the description as “BGP-15”) on the mitochondrial number as well as the expression of PGC-1α having key role in the mitochondrial biogenesis was investigated in the following tests.

1. Change of Mitochondrial Number in the Brown Adipose Tissue of Mice (In Vivo Test)

C57 Black mice having a body weight of 25-29 g were obtained from Charles River Breeding Ltd. The mice were kept in a daily cycle consisting of 12 hours' light, 12 hours' darkness at 22-25° C. and 50-70% relative humidity. The animals were fed with laboratory chow and tap water ad libitum. The animals were treated with a daily dose of 20 mg/kg of the compound “BGP-15” for 5 days. On day 6, the animals were sacrificed, and the brown adipose tissues were removed for further investigation.

Isolation of DNA: pieces of the brown adipose tissue were digested with proteinase-K enzyme at 37° C. for 18 hours, then the DNA was isolated. The purified DNA was amplified using genomic (nuclear) DNA primers (acting forward primer 5′-CCA-ACT-GGG-ACG-ACA-TG-3′ and reverse primer 5′-CCT-CGT-AGA-TGG-GCA-CA-3′, respectively) and, based on the result obtained, the samples were adjusted to identical nuclear DNA concentration. Then, PCR amplification was carried out using mitochondrial DNA probes to determine the amount of mitochondrial DNA. The following primers were employed in the amplification of mitochondrial DNA: 5′-AAA-GGT-TTG-GTC-CTG-GCC-TT-3′ and 5′-AAA-GGT-TTG-GTC-CTG-GCC-TT, respectively. The PCR products were separated by electrophoresis, and quantitatively determined based on the optical density after staining with Cyber green.

Owing to the treatment with the compound “BGP-15”, the ratio of mitochondrial/genomic DNA increased by 40%. This means that during the in vivo test, the number of mitochondria increased considerably in the brown adipose tissue, thus, the treatment with the compound “BGP-15” induced mitochondrial biogenesis.

2. Change of Mitochondrial Number in Rat Brown Adipose Cell Culture (In Vitro Test)

As an alternative methodical approach a specific mitochondrial fluorescent probe was used including the fluorescent stain Mito Tracker. A fluorescent microscope was employed to detect the stain that accumulated in the mitochondrion.

The brown adipose precursor cells were isolated from the brown adipose tissue present between the scapulas of 1-2 days old Sprague-Dawley rats. After digestion with collagen, the cells were suspended in DMEM (Dulbecco's modified Eagle's medium) culture medium (GibcoBRL, Eggenstein, Germany) supplemented with the following components: 10% of fetal calf serum (FCS, Gibco BRL, Eggenstein, Germany), 17 μM of D-pantothenic acid, 33 μM of d-biotin, 100 μM of ascorbinic acid, 1 μM of octanoic acid, 100 NE/ml of penicillin, 0.1 mg/ml of streptomycin, and 50 nM of insulin. Then, the cells were transferred into Petri dishes and grown at 37° C. in an atmosphere containing 5% of carbon dioxide and 95% of air. When the growing surface has been already covered by the cells, 1 μM of dexamethasone [(11β,16α)-9-fluoro-11,17,21-trihydroxy-16-methylpregna-1,4-diene-3,20-dione] and 1 nM of T3 were added to the culture medium to induce the differentiation of adipose cell. Then, treatment was carried out with the compound “BGP-15” at a concentration of 30 μM and the result of the treatment was examined after 6 days.

The cells were incubated with 100 nM of the fluorescent stain Mito Tracker at 37° C. for 30 minutes. The stain cumulating in the active mitochondria exhibits a fluorescent emission at 516 nm following the excitation at 490 nm. A fluorescence microscope Zeiss-Axioskop was used for the measurement at a magnification of 200 and 400 times. Exposures were prepared with a Nicon Coolpix 995 digital camera using identical exposure time, diaphragm aperture and digital picture size.

The evaluation of the exposures was carried out by means of a UTHSCA Image Tool Version 3.0 computer program by the examination of three confluent cell layers selected randomly. The results indicated that, owing to the 6 days' treatment, the mitochondrial number increased by 50% in relation to the starting amount.

Thus, in the above experiment, the mitochondrial biogenesis taking place in brown adipose cells of rat owing to the treatment with compound “BGP-15” could be confirmed using a further in vitro method.

3. Change of Mitochondrial Number in Rat Brain Endothelial Cell Culture (In Vitro Test)

In this test, the mitochondrial biogenesis was investigated in a culture of a still further cell type. In addition to the staining of mitochondria, the amount of COX-IV—a protein having key role in oxidative phosphorylation—was determined, too. This protein being the last element of the electron transport chain is responsible for the transformation of oxygen into water. The protein was detected by means of Western blot.

Rat brain capillary endothelial cell was grown in F12 culture medium (GibcoBRL, Eggenstein, Germany) supplemented with 10% of fetal calf serum, 400 IU/ml of penicillin, 50 μg/ml of streptomycin and 2 mM of glutamine. The cell culture was grown at 37° C. in an atmosphere containing 5% of carbon dioxide and 95% of air. The cells were regularly passed twice weekly. A culture forming a confluent cell layer was used for each experiment. The cells were treated with a concentration of 10 or 30 μM of the compound “BGP-15”, and the effect of the treatment was examined after 6 days. The culture medium was changed regularly, and, after 6 days, a part of the cells was examined by fluorescence microscopy described in chapter 2, while another part of the cells was analysed by Western blot as follows:

The cells were rinsed and collected in ice-cold PBS (phosphate buffer in physiological saline) (pH=7.4) containing 5 mM of ethylenediaminetetraacetic acid (EDTA), 5 mM of sodium fluoride and 100 μM of Na₃VO₄. Lysis of the cell pellet was carried out on ice under weak shaking for 10 minutes in a buffer solution containing 250 mM of sodium chloride, 50 mM of HEPES [4-(2-hydroxyethyl)-1-piperazineethanesulfonic acid] (pH=7.4), 1 mM of EDTA, 1 mM of EGTA [ethyleneglycol-bis(2-aminoethyl ether)-N,N,N′,N′-tetraacetic acid], 1.5 mM of magnesium chloride, 0.1% of Nonidet P-40, 40 mM of β-glycerol phosphate, 1 mM of Na₃VO₄, 1 mM of phenylmethyl-sulfonyl fluoride, 10 mM of benzamidine, 20 mM of NaF, 10 mM of sodium pyrophosphate, 10 μM/ml of aprotinin, 10 μg/ml of leupeptin, and 10 μg/ml of antipain. The insoluble cell debris was removed by centrifugation (13000 g, 12 minutes, +4° C.). The clear supernatant was admixed to ½ volume of 2× Laemmli gel loading buffer, the samples were boiled for 3 minutes, then maintained at −20° C. before use. The protein concentration was determined by means of Bio-Rad D_{c} (Protein Assay reagent (Bio-Rad Laboratories, Hercules, Calif., USA). Samples containing identical amount of protein were separated by polyacrylamide gel electrophoresis in the presence of 10% of sodium dodecylsulfate (10% SDS-PAGE) and blotted on PVDF membrane using a Trans-Blot SD Blotting Kit (Bio-Rad Laboratories). The imune detection was carried out by means of monoclonal anti-COX-IV antibody (A21348, Molecular Probes). Anti-mouse IgG HRP conjugated antibody (Cell Signalling Technologies) was used as secondary antibody. For the detection, ECL Plus System (Amersham) was employed. The spots formed were measured by densitometry to obtain optical density values which were averaged.

The results obtained by using the fluorescent stain indicated that treatment with 10 μM of the compound “BGP-15” increased the mitochondrial number by 50% and 30 μM of the compound increased the mitochondrial number by 130% as compared to the starting amount in rat brain endothelial cells after 6 days. The COX-IV protein content of the cells increased by 10% and 50%, respectively, due to treatments with the above concentrations of the compound “BGP-15” in 6 days in relation to the starting value.

Thus, in this experiment the mitochondrial biogenesis was shown in another cell type as a consequence of the treatment with the compound “BGP-15”. Detection was carried out by two independent methods: (a) fluorescence microscopy and (b) determination of the protein COX-IV being specific for the mitochondrion by Western blot.

4. Dependence of Mitochondrial Biogenesis on Time in HeLa Human Cervical Carcinoma Cell Line (In Vitro Test)

The human cervical carcinoma cells HeLa were grown in DMEM culture medium (GibcoBRL, Eggenstein, Germany) supplemented with 10% of fetal calf serum, 400 IU/ml of penicillin, 50 μg/ml of streptomycin and 2 mM of glutamine. The cell culture was grown at 37° C. in an atmosphere containing 5% of carbon dioxide and 95% of air. The cells were regularly passed twice weekly. For each experiment a confluent culture forming a continuous cell layer was used. To the cell cultures, the compound “BGP-15” was added in a concentration of 10 μM, and the effect of the treatment was examined from the second day until the fifth day following the treatment. The culture medium was regularly changed, and the cells were examined by fluorescence microscopy described in chapter 2. The mitochondrial number for days 2-5 in comparison with the starting value is given in Table 1. The control group was not treated with the compound “BGP-15” during growing.

TABLE 21
Dependence of mitochondrial biogenesis on time in HeLa cell line

| | Amount of mitochondria | |
|---|---|---|
| | in relation to the | |
| | starting value | |
| Time after | | 10 μM of |
| treatment in days | Control | ,,BGP-15” |
| 2 | 1.0 | 1.1 |
| 3 | 1.0 | 1.3 |
| 4 | 1.1 | 1.6 |
| 5 | 1.1 | 1.5 |

From Table 1 it can be seen that, on days 4 and 5, in the control group the mitochondrial number increased by merely 10%, while in the group treated with the compound “BGP-15” the mitochondrial genesis was of 50-60% in the cell line used.

5. Change of Mitochondrial Number in SHSY-5Y Human Neuroblastoma Cell Culture (In Vitro Test)

The aim of the test was, in addition to using a still further cell culture, to examine the induction of PGC-1α, the key transcription factor of mitochondrial biogenesis.

The human neuroblastoma cells grown in DMEM culture medium (GibcoBRL, Eggenstein, Germany) supplemented with 10% of fetal calf serum, 400 IU/ml of penicillin, 50 μg/ml of streptomycin and 2 mM of glutamine. The cell culture was grown at 37° C. in an atmosphere containing 5% of carbon dioxide and 95% of air. The cells were regularly passed twice weekly. To the cell cultures, the compound “BGP-15” was added in a concentration of 10 and 30 μM, respectively, and the effect of the treatment was examined after 6 days. The culture medium was regularly changed, and, after 6 days, the cells were examined by fluorescence microscopy described in chapter 2 or an analysis by Western blot was carried out as described in chapter 3 with the difference that in the immune detection, of course, polyclonal anti-PGC-1α antibody (A21348, Molecular Probes) was employed.

The results obtained in using the fluorescent stain indicated that treatment with 10 μM of the compound “BGP-15” increased the mitochondrial number by 60% and 30 μM of the compound increased the mitochondrial number by 170% in relation to the starting amount in SHSY-5Y human neuroblastoma cells after 6 days. Due to the treatments with the compound “BGP-15”, the PGC-1α level increased by 120 and 90%, respectively, as compared to the starting value.

In the latter test, the effect of the compound “BGP-15” on the mitochondrial biogenesis was proved in a further cell line. This effect was confirmed by an evaluation using fluorescence microscopy as well as the determination of the important transcription factor (PGC-1α) of mitochondrial biogenesis.

Based on the above test data it is evident that mitochondrial biogenesis is induced by treatment with the compound “BGP-15”. This treatment can be useful in states or diseases in which the mitochondrial number is not sufficient in comparison with the required amount. Such states or diseases include tissue regeneration, strengthening of weakened organism, diseases accompanied by loss of weight, regeneration phase of anorexia etc.

The considerable increase of the mitochondrial number can be advantageous in promoting fast muscle development or growth during muscle developing trainings, promoting muscle regeneration, promoting the physical condition of the body, even treatment of muscular strain and adaptation to high-altitude. The increase of the mitochondrial content of brown adipose tissue enhances the ability of the organism to lose superfluous energy independently of physical work.

Since ageing of mammals including man is accompanied by the frequency of the mutation of mitochondrial DNA [Coral-Debrinski, M. et al., Nature Genet., 2, 324 (1992)] and there are proofs that the deterioration of mitochondrial DNA and function participate in ageing [Trifunovich, A. et al., Nature, 429, 417 (2004)], the increase of the mitochondrial number can moderate the process leading to senescence. (In this context, senescence is considered as the condition of ageing which is often marked by a descrease in physical and mental abilities.)

Thus, O-(3-piperidino-2-hydroxypropyl)nicotinic amidoxime or a pharmaceutically suitable acid addition salt thereof and the pharmaceutical composition containing it can be used for, among others, increasing the mitochondrion content of brown adipose tissue, facilitating a fast growth and regeneration of muscle, improving the physical condition of the body and as a roborant.

The invention includes a method of treatment in which a mammal including man is treated with an effective non-toxic amount of O-(3-piperidino-2-hydroxypropyl)nicotinic amidoxime or a pharmaceutically suitable acid addition salt thereof to increase the mitochondrial genesis.

The pharmaceutical composition employed according to the invention may include any dosage form, however, it is suitable, primarily, for peroral administration, and can be solid or liquid.

The solid pharmaceutical compositions suitable for peroral administration may be powders, capsules, tablets, film-coated tablets, microcapsules etc., and can comprise binding agents such as gelatine, sorbitol, poly(vinylpyrrolidone) etc.; filling agents such as lactose, glucose, starch, calcium phosphate etc.; auxiliary substances for tabletting such as magnesium stearate, talc, poly(ethylene glycol), silica etc.; wetting agents such as sodium laurylsulfate etc. as the carrier.

The liquid pharmaceutical compositions suitable for peroral administration may be solutions, suspensions or emulsions and can comprise e.g. suspending agents such as gelatine, carboxymethylcellulose etc.; emulsifiers such as sorbitane monooleate etc.; solvents such as water, oils, glycerol, propylene glycol, ethanol etc.; preservatives such as methyl p-hydroxybenzoate etc. as the carrier.

The pharmaceutical composition contains dosage unit, in general. A typical dose for adult patients amounts to 0.1 to 1000 mg, preferably 1 to 250 mg of O-(3-piperidino-2-hydroxypropyl)nicotinic amidoxime or a pharmaceutically suitable acid addition salt thereof calculated for 1 kg body weight, daily. The daily dose can be administered in one or more portions. The actual dosage depends on many factors and is determined by the doctor.

Dosage forms listed above as well as other dosage forms, the manufacture thereof and pharmaceutical carriers are known from the literature, see e.g. Remington's Pharmaceutical Sciences, 18th Edition, Mack Publishing Co., Easton, USA (1990).

1. Use of O-(3-piperidino-2-hydroxypropyl)nicotinic amidoxime or a pharmaceutically suitable acid addition salt thereof for the preparation of a pharmaceutical composition increasing the mitochondrial genesis.

2. The use of claim 1 for the preparation of a pharmaceutical composition increasing the mitochondrion content of brown adipose tissue.

3. The use of claim 1 for the preparation of a pharmaceutical composition facilitating a fast growth and regeneration of muscle.

4. The use of claim 3 for the preparation of a pharmaceutical composition improving the physical condition of the body.

5. The use of claim 1 for the preparation of a roborant composition.

6. The use of claim 1 for the preparation of a pharmaceutical composition moderating the process leading to senescence.

7. A method for increasing the mitochondrial genesis in which the patient being in need thereof is treated with an effective amount of O-(3-piperidino-2-hydroxypropyl)nicotinic amidoxime or a pharmaceutically suitable acid addition salt thereof.
